(19) United States
(12) Patent Application Publication
(10) Pub. No.: US 2023/0405872 A1
(43) Pub. Date: Dec. 21, 2023

(54) MONITORING THE QUALITY OF CURING CONCRETE SYSTEMS WITH ELECTRICAL IMPEDANCE ANALYSIS

(71) Applicant: THE BOARD OF REGENTS FOR THE OKLAHOMA GRICULTURAL AND MECHANICAL COLLEGES, Stillwater, OK (US)

(72) Inventors: Lichun Chen, Stillwater, OK (US); Tyler Ley, Stillwater, OK (US)

(73) Assignee: THE BOARD OF REGENTS FOR THE OKLAHOMA GRICULTURAL AND MECHANICAL COLLEGES, Stillwater, OK (US)

(21) Appl. No.: 18/035,482

(22) PCT Filed: Nov. 17, 2021
(86) PCT No.: PCT/US2021/059733

§ 371 Date: May 4, 2023

Related U.S. Application Data
(60) Provisional application No. 63/114,719, filed on Nov. 17, 2020.

(51) Int. Cl. B28B 17/00; G01N 27/02; B28B 11/24

(52) U.S. Cl. CPC B28B 17/0081; G01N 27/021; B28B 11/245

(57) ABSTRACT

A system and method monitoring the properties of curing concrete systems to assist in adjusting curing conditions to produce predetermined properties in the hardened or cured concrete. The apparatus and method achieves this by providing a low-cost, minimally invasive means for gaining insight into the conditions of the curing concrete by using electrical impedance analysis. Sensors are distributed throughout the system to collect and transmit electrical impedance data, which is then interpreted in reference to correlations with other relevant physical and chemical properties.

### CROSS REFERENCE TO RELATED APPLICATION

This application claims the benefit of U.S. Provisional Patent Application No. 63/114,719 filed on Nov. 17, 2020, which is hereby incorporated by reference.

### FIELD

The present disclosure relates to systems and methods for the monitoring of chemical and physical properties using electrical impedance analysis and is particularly applicable in monitoring the quality of curing concrete systems.

### BACKGROUND

Concrete derives its strength from calcium silicate hydrate, a product of the hydration reaction that takes place when water is mixed with cement. A process known as curing, which involves providing continuous moisture and maintaining or providing heat to the concrete mixture, promotes the formation of calcium silicate hydrate. Curing conditions can impact the development of the calcium silicate hydrate microstructure. This in turn affects the properties of the cured concrete, such as porosity, permeability, shrinkage, creep, and strength.

To optimize curing conditions, it is beneficial to continuously monitor the conditions of the concrete mixture at various depths to ensure that desirable levels of moisture exist throughout the mixture. This kind of real-time monitoring is difficult to achieve without removing a portion of the curing mixture for testing, which takes time and is destructive to the final concrete structure. Also, these destructive tests measure at one point in time and do not provide insight into the continuous changes of the material. For example, curing conditions can be determined by removing a portion of the structure at a point in time and measuring the porosity, diffusion coefficient, or the degree of saturation at different depths but this is only done after the structure is complete.

A need exists for continuous, real-time monitoring of curing concrete conditions in order to modify the curing process to ensure certain properties are obtained in the concrete.

### BRIEF DESCRIPTION OF THE DRAWINGS

FIG. 1 is a schematic diagram of a system according to embodiments where impedance is used to determine physical properties.

FIG. 2 is a flow diagram of an embodiment of a method for using electrical impedance data to optimize chemical or physical properties of a curing concrete system.

FIG. 3 schematically portrays perspective and cross-sectional views of an example configuration of a mortar sample for impedance measurement.

FIG. 4 portrays schematic diagrams of (a) a mold for use in a splitting tensile strength test and (b) a mortar sample undergoing a splitting tensile strength test.

FIG. 5 portrays a graph displaying the average resistivity over depth with respect to time for different curing methods.

FIG. 6 portrays a graph displaying the percent moisture change over time for different curing methods.

FIG. 7 portrays graphs displaying resistivity gradation profiles at different time points for different curing methods.

FIG. 8 portrays graphs displaying the porosity and degree of saturation gradation profiles over the sample depth at 12, 24, 48, 72, and 96 hours of difference curing methods.

FIG. 9 portrays graphs displaying the resistivity and degree of saturation at 12, 24, 48, and 72 hours of hydration for different curing methods.

FIG. 10 portrays a graph displaying the degree of saturation as a function of resistivity at 19 mm and 57 mm from the surface after hydrating for 12, 24, 48, and 72 hours, respectively.

FIG. 11 portrays a graph displaying the predicted degree of saturation from resistivity at 12, 24, 48, and 72 hours of hydration compared with the measured degree of saturation.

FIG. 12 portrays a graph displaying the relationship between porosity and resistivity at 86% degree of saturation.

FIG. 13 portrays a graph displaying the splitting tensile strength at 72 hours and the average resistivity along depth at 72 hours for the three curing methods.

FIG. 14 portrays a graph displaying the diffusion coefficient for various curing methods as it relates to the average resistivity over depth at 72 h. This data was obtained for a concrete mixture. The time next to the individual data points is the time when the wet curing was applied to the surface of the samples. The samples were cured under a heat lamp to increase the moisture loss of the samples.

FIG. 15 is a graph illustrating the relationship of moisture content, resistivity, and temperature for a concreate mixture.

### DETAILED DESCRIPTION

The present disclosure may be understood more readily by reference to these detailed descriptions. Numerous specific details are set forth in order to provide a thorough understanding of the various embodiments described herein. However, it will be understood by those of ordinary skill in the art that the embodiments described herein can be practiced without these specific details. In other instances, methods, procedures, and components have not been described in detail so as not to obscure the related relevant feature being described. Also, the description is not to be considered as limiting the scope of the embodiments described herein. The drawings are not necessarily to scale and the proportions of certain parts have been exaggerated to better illustrate details and features of the present disclosure.

Before discussing the presently disclosed inventive concepts in detail by way of exemplary description, drawings, and example, it is to be understood that the inventive concepts disclosed herein are not limited in application to the details of construction and the arrangement of the compositions, formulations, steps, or components set forth in the following description or illustrated in the drawings and/or examples. The presently disclosed inventive concepts are capable of other embodiments or of being practiced or carried out in various ways. As such, the language used herein is intended to be given the broadest possible scope and meaning; and the embodiments are meant to be exemplary, not exhaustive. Also, it is to be understood that the phraseology and terminology employed herein is for the purpose of description and should not be regarded as limiting except where indicated as such.

All of the compositions, devices, systems, and/or methods disclosed herein can be made and executed without undue experimentation in light of the present disclosure. Although certain steps are described herein and illustrated in the figures as occurring sequentially, some steps may occur simultaneously with each other or in an order that is not depicted.

As utilized in accordance with the present disclosure, the following terms, unless otherwise indicated, shall be understood to have the following meanings: the use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” As used in this specification and claims, the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Throughout this disclosure, the terms “about” and “approximate,” and variations thereof, are used to indicate that a value includes the inherent variation or error for the device, system, the method being employed to determine the value, or the variation that exists among the study subjects.

The present disclosure relates to systems and methods for the monitoring of chemical and physical properties using electrical impedance analysis, particularly as it pertains to concrete curing.

### Definitions

Concrete: a composite material composed of fine and/or coarse aggregate bonded together with a cement that hardens (cures) over time. The aggregate generally comprises sand, gravel, and/or crushed stone. More typically, the aggregate consists essentially of one or more of sand gravel, or crushed stone. A mortar is a special type of concrete that does not contain larger aggregates such as gravel, and/or crushed stone.

Cement: a substance that sets, hardens, and adheres to other materials to bind them together, often lime or calcium silicate based but other types of cement are possible. The cement can be a hydraulic or non-hydraulic cement. Generally, the cement referred to herein will be a hydraulic cement, typically a portland cement. However, in some embodiments the cement can be siliceous fly ash, calcareous fly ash, slag cement or silica fume, or a Portland cement blended with one or more of the foregoing. It is also possible to use a calcium aluminate, or calcium sulfoaluminate, geopolymer, or magnesium based cement.

Curing: the process by which cement forms a hardened structure over time, which usually occurs as the result of the hydration reaction that occurs when water is mixed with the cement and a certain temperature is maintained.

Impedance/Electrical Impedance: the measure of a substance's opposition to the flow of alternative electric current. As used herein, the term “impedance” or “electrical impedance” also includes less complex resistance measurements, such as electrical resistance, which is the resistance offered by a substance per unit length for a unit cross-section. In simple terms, impedance possesses both magnitude and phase, and resistance has only magnitude. As used herein, references to electrical impedance analysis also include electrical resistivity analysis, and tests, sensors, and other systems referring to electrical impedance also include those utilizing electrical resistivity. For example, an electrical impedance sensor also includes electrical resistivity sensors. For example, measuring the electrical impedance across all or a portion of a substance also includes measuring the electrical resistivity across all or a portion of a substance.

Moisture content: the amount of non-chemically bound water in the concrete. This is expressed by a term called the degree of saturation. The degree of saturation is the ratio of the moisture content of the sample in the current state to a sample that has been dried in an oven just above 100° C. to remove all non-chemically bound water.

Porosity: the measure of the volume of the voids in concrete.

Permeability: a general term used by the concrete industry to mean the ease of penetration of outside chemicals such as water or brine solutions such as deicer salts or salt water. This can also be air penetrating the concrete. In this work, the diffusion coefficient is a more specific way to determine permeability. The methods to measure this are outlined in previous publications.

Strength: The ability to resist external or internal loading. This may be compressive, shear, flexural, or tensile strength. In this work, the tensile strength of the concrete is measured with a splitting tensile strength test.

The purpose of curing is to promote chemical reactions to build the microstructure of the concrete. The microstructure is essential to properties such as porosity, permeability, strength, shrinkage, and creep. As a result, it is valuable to have a low-cost method that can be used to evaluate the effectiveness of curing.

The present disclosure takes advantage of a discovery relating to correlations among the physical properties of concrete systems undergoing different curing methods. One of the unexpected results is that the moisture content and other properties in a curing concrete mixture may be instantaneously quantified during the early stages of the reaction. Tests measured the electrical impedance at various depths within curing concrete samples undergoing dry curing, sealed curing, and wet curing. The gradient in the electrical signal correlates to the gradient in the moisture of the samples. Because the moisture content is different at different depths, the properties of the concrete also vary with depth. Various properties of the samples, including porosity, degree of saturation, rate of heat exchange, diffusion coefficient and tensile strength, were compared with the electrical impedance data. A strong correlation was found. One of the unexpected results is that electrical impedance provides a quantitative measurement giving meaningful insight into the characteristics of curing concrete mixtures. Particularly, electrical impedance has the potential to examine curing methods and the corresponding impacts on the concrete microstructure, strength, and other properties.

The systems and methods disclosed herein provide an economical and low cost means for obtaining rapid and continuous electrical impedance data. Unlike previous techniques, the systems and methods disclosed herein accomplish this without the need to remove or otherwise deface or destroy a portion of the curing concrete mixture. Also, the method examines the gradient within the sample by examining at different depths from the surface. This configuration is very important as it allows moisture loss or property development to be compared at different depths in the sample. The use of the gradient to monitor the change in performance also allows the testing to be done without calibration to the impedance and material properties of the mixture because the method focuses on the gradient within the sample. The systems and methods disclosed herein are applicable to small-scale concrete constructions and projects, such as landscaping or various paving applications, as well as large-scale or industrial projects, including the construction of bridges, roads, tunnels, and other buildings and structures.

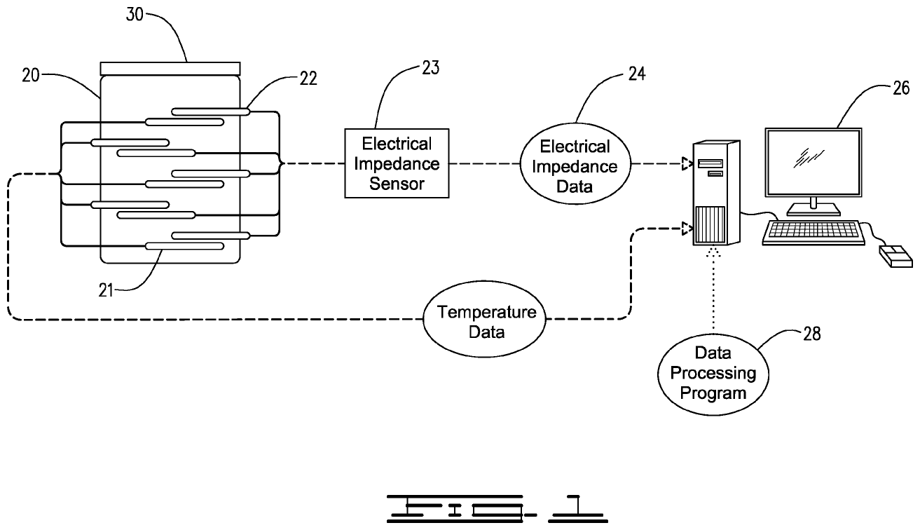

In one embodiment, as portrayed in FIG. 1, the system comprises a concrete mixture 20. Typically, the concrete mixture will start in an uncured state or partially cured state. The apparatus further comprises one or more temperature sensors (such as thermocouples 21) and one or more electrical impedance sensors 23 that are placed in close proximity to correlate their signals. The sensor arrangement is schematically illustrated in FIG. 1. It should be understood that generally there will be a sensor 23 and sensor 21 at each location to produce temperature and impedance measurements for that location. It is within the scope for sensor 23 and sensor 21 to be combined into a single sensor.

The sensors 23 comprising a plurality of electrically conductive components 22 distributed at one or more respective locations within the concrete mixture 20 so as to pass an electrical current across respective portions of the concrete mixture. These sensors are arranged at known depths to determine an electrical impedance gradient within the concrete mixture. Sensor 23 is configured to produce electrical impedance data 24 based on the electrical current flow through the concrete mixture. Sensors 23 may detect the electrical impedance across respective portions of the concrete mixture and convert the electrical conduction responses into electrical impedance data 24.

The temperature sensors measure temperature at the same location as electrical impedance sensors 23. Temperature of the concrete mixture will impact the measured resistivity or electrical impedance. This placement of the sensors allows correction of the impedance/resistivity data based on the temperature at the location of the sensors, as further discussed below.

The one or more electrical impedance sensors 23 connect to a central processing unit (“CPU”) 26 so as to transmit electrical impedance data 24 to the CPU 26. For example, CPU 26 may be a desktop PC and may work in conjunction with one or more of a main memory configured to store computer readable code, at least one processor coupled to the main memory to execute the computer readable code in the main memory, and may work in tandem with a screen display, input and output devices, and other necessary computer applications and accessories. A data processing program 28 works in conjunction with CPU 26 so as to process, record, and/or display electrical impedance data 24. The thermocouple 21 also transmits temperature data to the CPU 26 for adjusting the impedance measurement for temperature.

In one embodiment, electrically conductive components 22 may be stainless steel rods. In another embodiment, they may be any electrically conductive material and may vary in size, shape, number, and physical orientation, as determined by one skilled in the field. In embodiments, electrically conductive components may be distributed in the concrete mixture by first placing the components into position and then pouring the concrete mixture over the electrically conductive components. For example, the plurality of electrically conductive components may be distributed in the concrete mixture by first pouring the concrete mixture in place (such as in a mold or within a form), and then placing the components into the concrete.

In one embodiment, CPU 26 receives electrical impedance data 24 via a wired or wireless connection or connections. In another embodiment, data processing program 28 processes and/or displays electrical impedance data 24 continuously as it is transmitted to CPU 26. In another embodiment, data processing program 28 processes and displays electrical impedance data 24 in the form of graphs, charts, tables, or other user-friendly displays.

In another embodiment, data processing program 28 displays other various correlative physical properties, such as porosity, degree of saturation, rate of heat exchange, and the splitting tensile strength, with or separately from electrical impedance data 24. Correlations may be determined based on data obtained from prior laboratory analysis of curing concrete systems, such as by the method described in the example below. The correlations may vary depending on the given curing concrete composition and application. Correlations may additionally be derived or extrapolated, or both, as it occurs to one having ordinary skill in the art based on this disclosure. Data processing program 28 may have preexisting correlations for chemical and physical properties downloaded or inputted so that electrical impedance data 24 may be continuously processed and converted into meaningful data and data displays relating to the correlative properties.

In embodiments, the processing unit processes the electrical impedance data by a data processing program configured to perform one or more of the steps described in the below processes. For example, the following operational steps can be performed:
(a) defining at least one predetermined property of the curing concrete mixture or hardened concrete wherein the predetermined property includes one or more of a predetermined moisture content of curing concrete, predetermined porosity of the hardened concrete, a predetermined degree of saturation of the curing concrete, a predetermined degree of saturation of the hardened concrete, the diffusion coefficient of the hardened concrete, and a predetermined splitting tensile strength of the hardened concrete;
(b) correlating the electrical impedance data with a preexisting set of electrical impedance correlation data so as to determine at least one expected property of the curing concrete mixture or hardened concrete based on changes to the electrical impedance data over time, wherein the expected property includes one or more of the moisture content of curing concrete, porosity of the hardened concrete, degree of saturation of the curing concrete, the degree of saturation of the hardened concrete, the diffusion coefficient of the hardened concrete, and splitting tensile strength of the hardened concrete;
(c) determining if the expected property meets the predetermined property; and
(d) when the expected property does not meet the predetermined property, determining a change in the set of curing conditions which will result in a change to the expected property so as to meet the predetermined property.

In embodiments, a moisture material 30 can be placed in contact with part or all of the surface of the curing concrete mixture. For example, moisture material 30 can be a water absorbent material that is saturated or a water-impermeable material. If a saturated water absorbent material is used it can provide water to the concrete mixture to maintain moisture levels in the concrete, reduce the rate of moisture depletion in the concrete mixture or even temporarily increase the moisture content of the concrete mixture. In embodiments, moisture material 30 can be a water-impermeable material or a semipermeable material such as a spray-applied curing compound. This material seals part or all of the surface of the curing concrete mixture so as to minimize moisture loss by evaporation.

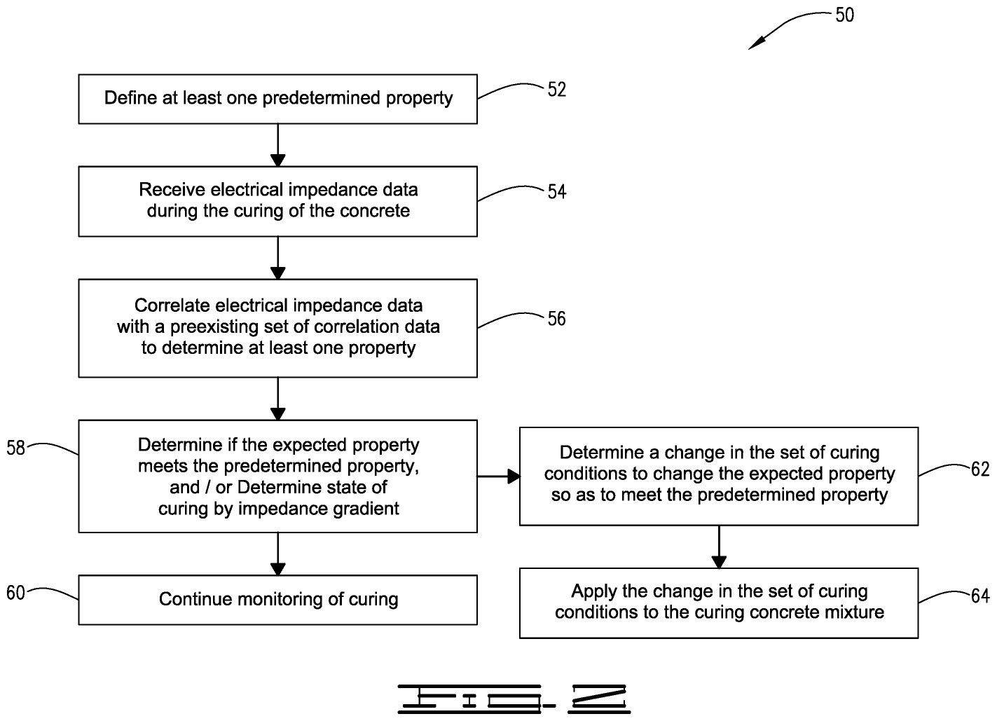

In one embodiment, as portrayed in FIG. 2, a method 50 for monitoring the physical and chemical properties of a concrete mixture is illustrated. In method 50, at least one predetermined property is optionally defined in step 52. The predetermined property is a physical property objective for the curing concrete based on a desired quality of the hardened concrete. For example, the predetermined property can be one or more specific physical properties desired during the curing concrete, such as a moisture content, a degree of saturation, or a rate of temperature change or heat change for the curing concrete. For example, the predetermined property can be one or more specific physical properties desired for the hardened concrete. In embodiments, the predetermined property for the hardened concrete can include one, two or all of porosity, degree of saturation, diffusion coefficient, and splitting tensile strength. Step 52 is an optional step in that some embodiments will not require that a predetermined property be defined but will determine the state of curing solely by the impedance gradient, as further discussed herein.

Additionally, in step 54, the process receives electrical impedance data representative of impedance across all or a selected portion of the curing concrete mixture. The electrical impedance data is received during the curing of the curing concrete mixture and is representative of changes in impedance over time. The electrical impedance data is representative of all or a selected portion of the curing concrete and is representative of the change in impedance over time. Once received, the electrical impedance data can be analyzed to determine an electrical impedance gradient with respect to the depth of the concrete. Generally, the electrical impedance data and/or gradient can be correlated with an amount of moisture loss at a plurality of locations within the concrete mixture during the time analyzed. For example, the data can be correlated with a preexisting set of electrical impedance correlation data in step 56. For example, the data or gradient can be correlated for moisture, porosity, degree of saturation, rate of heat change, and strength. For example, the electrical impedance correlation data can include data correlating electrical impedance to the porosity of hardened concrete, to the degree of saturation of the hardened and/or curing concrete; to the rate of heat change of the curing concrete, to the moisture content of the curing concrete, and/or to the splitting tensile strength of the hardened concrete. As will be realized, the correlation data can vary for concrete mixtures, and thus generally, the process will include selecting correlation data corresponding to the curing concrete mixture's composition.

The correlation of the electrical impedance data and/or gradient can include correcting for temperature across the locations of impedance measurements in the concrete mixture. The temperature of the concrete will impact the measured resistivity or electrical impedance. This can be corrected by measuring both the temperature and electrical impedance or resistivity at the same location. This is done by placing the two sensors close to one another. This will allow the temperature sensor to be used to correct for the predicted moisture level, porosity, strength or permeability. For example, the impedance data and/or gradient can be adjusted or corrected based on the difference in the measured temperature at two or more locations.

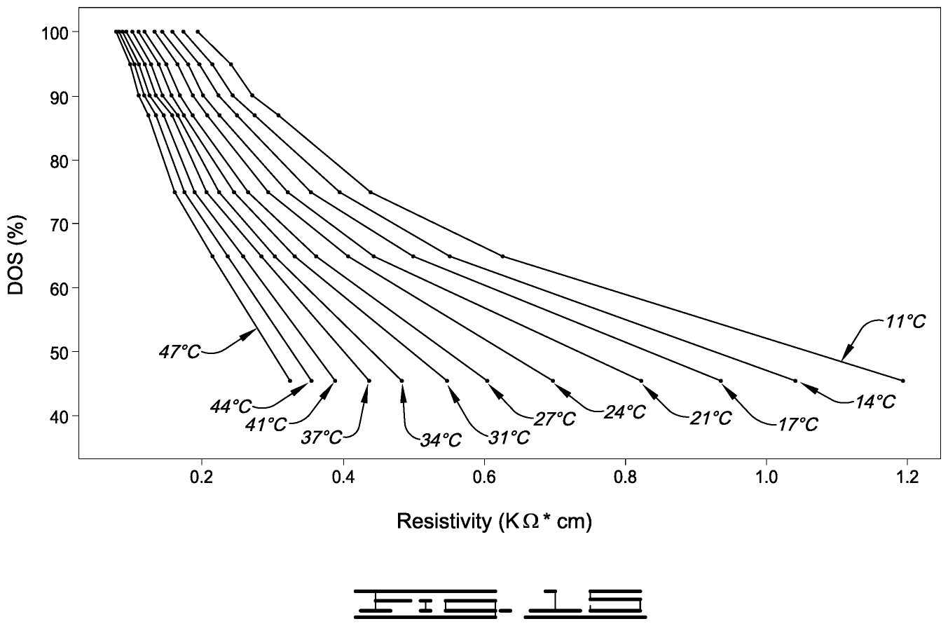

An example of this is shown in FIG. 15. This graph was created by making concrete samples and adjusting their temperature and moisture content and measuring the resistivity. This shows how the moisture content, resistivity, and temperature are related. These results show that a correction equation can be used to account for temperatures at different depths in the sample and determine how this impacts the resistivity of the sample. Since temperature sensors are used at different depths in the presented invention, then this will allow this correction to be made. Other temperatures besides the ones shown can be used with additional testing. These temperatures show a useful range of temperatures that may be experienced in practice.

In some embodiments, the correlation is used to determine if changes to curing need to be made in order to reduce the electrical impedance gradient for some or all of the remaining curing time. For example, the correlation can be with regard to a general reduction in the electrical impedance gradient, and/or can be with regard to how well the measured electrical impedance gradient matches a predetermined electrical impedance gradient. The predetermined electrical impedance gradient having been defined to be a gradient that correlates with concrete curing to have specific properties.

In some embodiments, the correlation is used to determine at least one expected property of the curing concrete or the hardened concrete based on changes to the electrical impedance data over time. Accordingly, the expected properties determined from the correlation typically parallel or are the same type as the predetermined properties; although not necessarily the same value. For example, the expected properties can include the current moisture content of the curing concrete, the current degree of saturation, or the current actual rate of temperature or heat change for the curing concrete, based on the correlation. Optionally, the expected properties can be the predicted moisture content, degree of saturation or rate of heat exchange for the curing concrete at some predetermined time in the future, based on the correlation. For example, the expected properties can include the current moisture content or the predicted moisture content at some predetermined time in the future, based on the correlation. For example, the expected properties can include one or more of porosity, degree of saturation, permeability, and splitting tensile strength for the hardened concrete.

When predetermined properties are utilized, once the expected properties are determined, the process can then determine if the expected property meets the predetermined property in step 58. This determination can be based on the expected property exactly matching the predetermined property, but more typically, it is based on the expected property being within a percentage of the predetermined property; for example, within 2%, 5%, 10% or 20% difference of the predetermined properties. In some instances, it can be sufficient that the expected property equals or exceeds the predetermined property, or that it does not exceed the predetermined property. For example, a predetermined strength may be the minimum necessary for the hardened concrete; thus, as long as the expected strength equals or exceeds the predetermined strength, the expected property is deemed to be acceptable.

If the expected property meets the predetermined property, then the process can continue monitoring the curing (step 60) by continuing with steps 54, 56 and 58 until either there is high confidence that the property has been obtained or the hydration reaction slows until the curing has little impact on the desired property. If the expected property does not meet the predetermined property, then at step 62 a change in the set of curing conditions can be made. The change in the set of curing conditions is configured to result in a change to the expected property so as to meet the predetermined property. For example, the set of curing conditions can include at least one of moisture content of the curing concrete mixture and rate of heat exchange of the curing concrete mixture. Where the electrical impedance gradient is correlated with a predetermined electrical impedance gradient, the change in the set of curing conditions can be configured to result in a change in the electrical impedance gradient so that it more closely matches the predetermined electrical impedance gradient. In some applications, this can be reducing the electrical impedance gradient, such that a second measurement of the gradient at a later time will be reduced from the measurement at an earlier time.

Next in step 64, the determined change is applied to the set of curing conditions to the curing concrete mixture so as to modify the curing of the concrete mixture and match the expected properties to the predetermined properties. For example, the modification can comprise adjusting the rate of moisture change in the curing concrete and/or adjusting the rate of heat change in the curing concrete. The rate of moisture change in the curing concrete may be adjusted by placing or removing, as required, an absorbent material saturated with water. For example, the rate of moisture change can be adjusted by a water-impermeable material for use in sealing the surface of the concrete mixture. For example, the rate of moisture change can be adjusted by leaving the curing concrete mixture open to the surrounding environment. As will be realized, generally the curing will continue to be monitored during and after steps 62 and 64. Typically, the monitoring will continue until there is high confidence that the property has been obtained or until the hydration reaction slows so that the curing has little impact on the desired property. In embodiments, certain of the steps may be performed in different order or simultaneously.

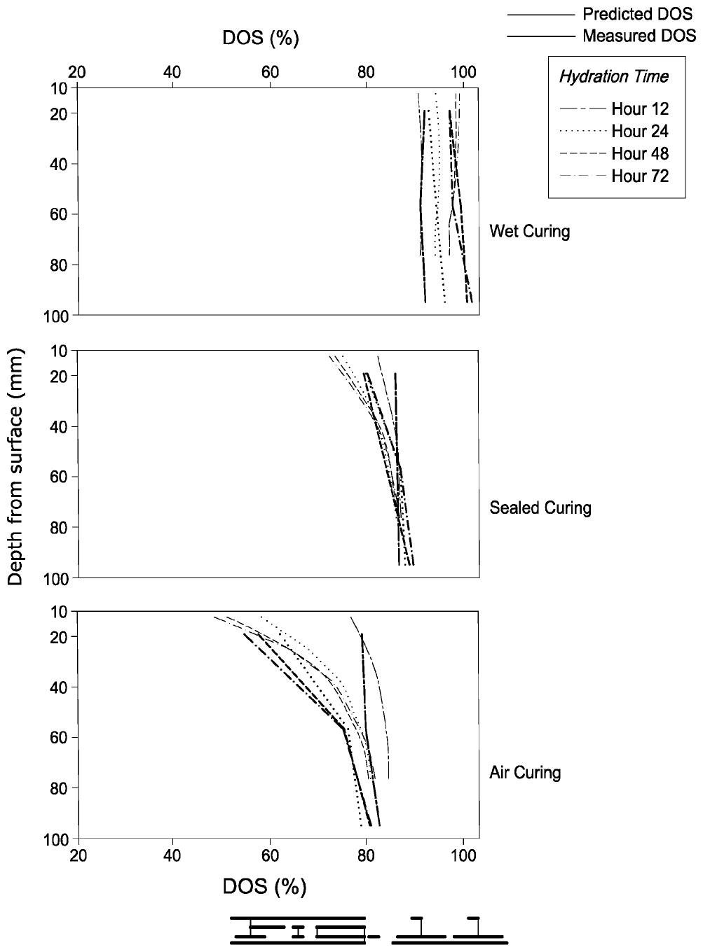

It is also possible to use the system to gain useful information without correlating the electrical impedance to a known property. Because measurements are made at different depths of the sample, the electrical impedance can be used to compare the signals at different depths to examine the uniformity of the hydration. Because more water is lost at the surface of the concrete from evaporation than the core, this will have a greater impact on the electrical impedance measurement at the surface. This means that a slope or gradient of the electrical impedance signal over the depth can be a useful tool to evaluate the quality of the curing. For example, FIG. 11 shows a large gradient between the top and bottom of the air cured concrete and almost no gradient in the wet cured concrete. The sealed cured concrete loses some water, which is likely in the air space above the sample, and so this has some impact on the measured gradient. This gradient is smaller than the one measured on the air cured sample. This method highlights that the top of the concrete is losing moisture. This may be a signal to the builders that the moisture material needs to be placed on the concrete and the device can guide them to make this adjustment.

This approach is useful because it does not require any correlation between physical properties and the electrical impedance signal. This approach may be done by examining the gradient of the signal and sending a warning if the evaporation rate is too high at the surface compared to the other depths of the sample. This can be implemented by either measuring the impedance in an actual concrete element or on a sample that is representative of the curing method. This approach could also be used to determine how susceptible a mixture is to loss of water in lab testing and how this impacts the properties of the hardened concrete. The electrical impedance can also be correlated to a known property for the mixture and this could be used in the field for acceptance of the concrete.

In embodiments, the CPU receives the electrical impedance and temperature data transmitted from the one or more of the sensors via a wired or wireless connection or connections. The data processing program may process and display the electrical impedance data continuously as it is transmitted to the CPU. In embodiments, the data processing program processes and displays the electrical impedance data in the form of graphs, charts, or other user-friendly displays. The data processing program may display the electrical impedance data together with other various correlative physical properties, such as moisture content, porosity, degree of saturation, rate of heat exchange, permeability, the splitting tensile strength, or any other correlated property. In embodiments, the electrical impedance data is collected and interpreted in conjunction with the various other correlative physical properties based on correlations that have been derived, extrapolated, measured, or otherwise determined with respect to curing concrete systems having similar properties and compositions as the curing concrete mixture.

In embodiments, system characteristics may be adjusted by altering the moisture provided to the curing concrete system. A variety of curing techniques may be employed to do so. In wet curing, moisture may be continuously provided at the surface of the curing concrete system by bringing a water-saturated absorbent material into contact with the surface of the curing concrete. In sealed curing, moisture may be retained by sealing off the curing concrete system with an impermeable or semi permeable material so as to minimize moisture loss by evaporation. In dry curing, moisture may be released over time by exposing the surface of the curing concrete system to the environment. In embodiments, a variety of correlations may exist depending on the type of curing used. Depending on the type of curing used, correlations between electrical impedance and properties such as porosity, degree of saturation, rate of heat exchange of the curing concrete, permeability, or splitting tensile strength of the cured concrete may vary. In embodiments, correlations between given properties under similar conditions and curing methods inform the interpretation of electrical impedance data. This makes the invention disclosed herein a valuable means for determining the quality of curing concrete samples, which helps in the field to guide curing practices. For example, based on the impedance gradient in a concrete sample, curing conditions may be modified, such as by switching to wet, sealed, or dry curing, to ensure that the impedance always stays below some critical value, as determined with respect to a given application.

### Example

The following example is merely illustrative and not limiting of the present disclosure. While the following example describes a concrete sample tested in a mold, it is to be understood that the present disclosure is more broadly applicable to any kind of curing concrete structure, whether in a permanent, reusable mold, in makeshift molds or casts for large-scale projects, or in free-standing or reinforced curing concrete mixtures.

In this example, the concrete mixture was a mortar mixture, prepared in accordance with ASTM C305, and had a water-to-cement ratio of 0.45. The mixture proportion by volume is shown in Table 1 below. The cement used met the requirements of an ASTM C150 Type I Portland cement. The fine aggregate used in the mortar mixture was natural sand meeting the requirements of ASTM C33. The Blaine of the cement was 3,560 cm²/g, and the free lime content was 1.4%. The chemical compositions of the mixture is shown in Table 2 below.

TABLE 1
Mixture Proportion by Volume of Mortar

| | Cement | Water | Fine Aggregate | Dry |
|---|---|---|---|---|
| Volume (%) | 18.5 | 23.4 | 56.7 | 0.4 |

TABLE 2
Chemical Composition of Cement

| | SiO₂ | Al₂O₃ | Fe₂O₃ | CaO | MgO | SO₃ | Na₂O | K₂O | C₃S | C₂S | C₃A | C₄AF | LOI |
|---|---|---|---|---|---|---|---|---|---|---|---|---|---|
| Cement | 21.1 | 4.8 | 3.1 | 64.5 | 2.33 | 3.2 | 0.17 | 0.58 | 50 | 23 | 7 | 9 | 2.6 |
| (mass %) | | | | | | | | | | | | | |

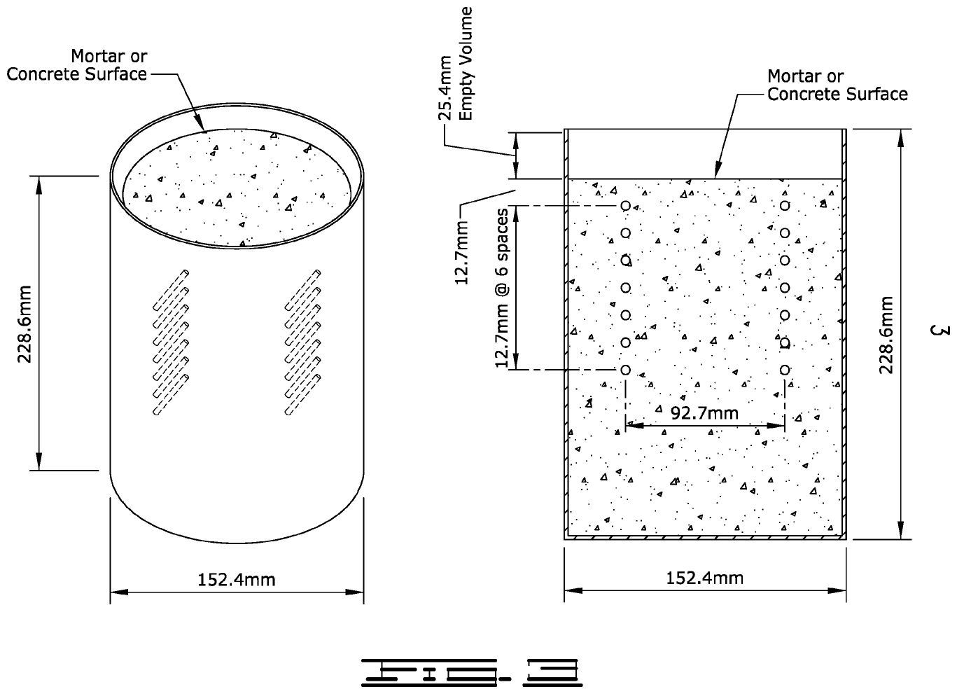

In accordance with the depiction in FIG. 3, the mortar mixture was poured into plastic cylinder molds having a diameter of 152.4 mm and a height of 228.6 mm. The cylinders had seven layers of 4-40 threaded stainless steel rods (2.8 mm in diameter and 40 threads per 25.4 mm). Each layer included two rods with a horizontal spacing of 92.7 mm and a vertical spacing of 12.7 mm. The steel rods were installed through the molds and stabilized and sealed with glue before the mortar was poured. This configuration is shown in FIG. 3.

The mortar filled the mold to 203.2 mm in height, the top 25.4 mm remaining empty to allow different curing methods to be applied. The mortar was poured in three layers, each layer being consolidated for 10 s with a vibrating table at a frequency of 60 Hz to remove entrapped dry air and promote a good bond with the threaded rod.

Wet curing, sealed curing, or dry curing were conducted on samples in an environmental chamber with a temperature of 23° C. and humidity of 50%. The curing methods were applied to the surface of the samples. In wet curing, three layers of water-soaked burlap were placed on the surface, and then the surface of the mold was sealed with aluminum tape. The burlap was removed and soaked with water every 24 hours and replaced on top of the sample. In sealed curing, the mold was covered with aluminum foil and sealed with aluminum tape to prevent moisture loss. The dry curing method left the sample open to the 50% humidity environment.

The resistivity was measured between the rods every 10 minutes through the hydration process. This may be measured more regularly or slowly depending on the application. Impedance is composed of two parts, real impedance, and imaginary impedance. The measurement of the real and imaginary impedance of a specimen would change depending on the excitation frequency of the chosen sensor. When a proper frequency range is chosen, the imaginary impedance part can be forced to zero and only the real impedance value was measured. At this point, the specimen is measured as a pure resistor and the real impedance is the resistivity of the specimen. In the proposed system, an excitation frequency of 30 kHz was used, which forced the imaginary part of the measurement to be zero. Hence, the resistance of the concrete sample is obtained, which is also called the bulk resistivity of concrete. A different frequency could be used in other applications. The circuit for the impedance measurement was programmed with the Arduino platform. An Arduino Mega 2560 was used as the central processor with a 12-Bit impedance converter AD5933. A frequency of 30 kHz was used. This was chosen because at this frequency the imaginary part of the impedance is close to zero. The calibration process calculated the Gain Factor of the system based on a known resistor. The system was calibrated with a 100-ohm resistor. Five multiplexers were used to measure 80 channels simultaneously. The results were recorded on an SD card and retrieved for analysis.

Since the frequency range selected forced the imaginary impedance to close to 0, the measured impedance was also called the bulk resistance of the mortar sample. The unit of the measured bulk resistance is ohms. To get the resistivity of the mortar sample, the following equation was used:

Resistivity=bulk resistance (Ω)*A (mm²)/L (mm) KΩ*cm.

In this equation, A was assumed to be the rectangular cross-sectional area of the electrode perpendicular to the signal. The area was taken as 338.8 mm², since the electrode was 2.8 mm in diameter and approximately 120.96 mm long. The distance between the electrodes, L, was 92.7 mm.

The mass of the wet, sealed, and air curing samples over the first 72 h of hydration was measured and recorded every hour. The percentage of mass change was then obtained with the following equation:

$$\% Mass Change = \frac{{M}_{n} - {M}_{i}}{{M}_{i}} \times 100 .$$

The porosity and degree of saturation were investigated to support the resistivity measurements. The porosity illustrates the microstructure development and the DOS shows the amount of moisture within the sample. The DOS is a critical factor in promoting early hydration within the sample. Other research shows that hydration ceases at a relative humidity of about 80% due to negative capillary pressure that opposes the reaction.

The mortar samples for the porosity and DOS measurement were cast into tubes with 25.4 mm diameter by 114.3 mm tall. Each tube was filled with three layers of mortar. The mortar was from the same mixture as the one for the resistivity measurement. The wet cured DOS samples were stored in a fog room with a constant humidity of 100% and temperature of 23° C. with its surface open to the environment. This was because the surface area of the container was too small to apply wet burlap. The sealed curing and air curing were conducted in an environment with 50% humidity and 23° C. temperature. In the sealed curing, the sample top was sealed with the lid for the tube.

The porosity and DOS were measured when the samples had hydrated for 12 h, 24 h, 48 h, 72 h, and 96 h. When the sample reached the designated hydration time, it was demolded and cut into 3 segments that were each 38.1 mm tall. This means that the distance to the midpoint of each segment was 19.1 mm, 57.2 mm, and 95.3 mm from the sample surface. This was done so that the porosity and DOS of each segment could be compared to the resistivity measurements at similar depths.

The porosity and DOS were determined by ASTM C642 with some minor changes where the samples were saturated within a vacuum chamber at pressure of 37 mmHg±5 mmHg instead of boiling in water. The equations below were used to calculate the porosity and degree of saturation, where IV, is the initial weight of the sample, WI is the oven dried weight at 110° C., Wₛₐ is the saturated surface dried (SSD) weight, and Wₛᵤ is the weight of the sample while the sample is suspended in water and is equal to the difference between the sample weight and its buoyant force:

$$Porosity = \frac{{W}_{s a} - {W}_{d}}{{W}_{s a} - {W}_{s u}} \times 100$$

$$DOS = \frac{{W}_{i} - {W}_{d}}{{W}_{s a} - {W}_{d}} \times 100.$$

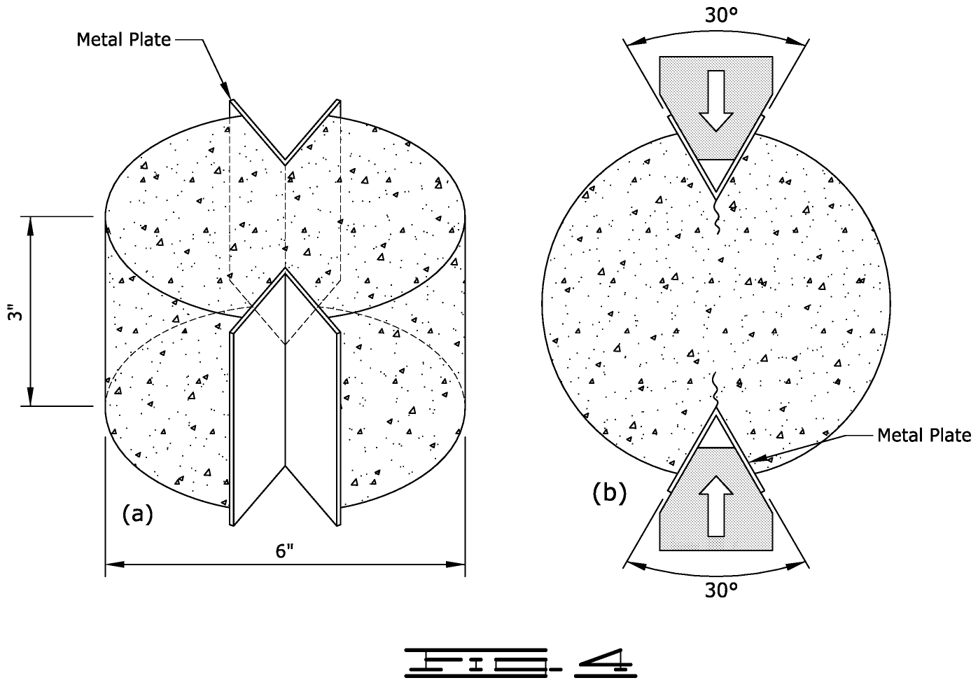

The mortar samples for splitting tensile strength were cast into 152.4 mm diameter by 76.2 mm tall plastic cylinders. Metal plates were fixed into the mold to form 2 notches with a 30° angle at the sample surface along its length shown in FIG. 4. The mold was filled in 2 layers and vibrated for 10 s on the vibration table at 60 Hz. The samples were cured the same way as the resistivity samples. After the mortar sample hydrated for 72 h, the sample was demolded and split with a hydraulic press as shown in FIG. 4. The loading platen had the same dimensions as the notches. The splitting test was conducted in this way because the sample failure is caused by the tension forces caused by the loading platen being pushed in compression and pushing on the angled inserts. The platen was loaded at a rate of 2000 N/min. Each curing method used 5 samples for this test. The samples that did not split at the notches were excluded from the strength calculation.

A diffusion test was conducted on mortar samples after 72 hours. The diffusion coefficient was defined as the amount of a particular substance that diffuses across a unit area in one second under the influence of a gradient of one unit. In this test, the diffusion coefficient of the mortar was obtained by placing KI solution on the sample top and monitoring the ion penetration depth over time. The higher the diffusion coefficient, the easier for the sample to be penetrated by chemicals, and thus the more susceptible the sample was to damage caused by chlorides or other chemicals.

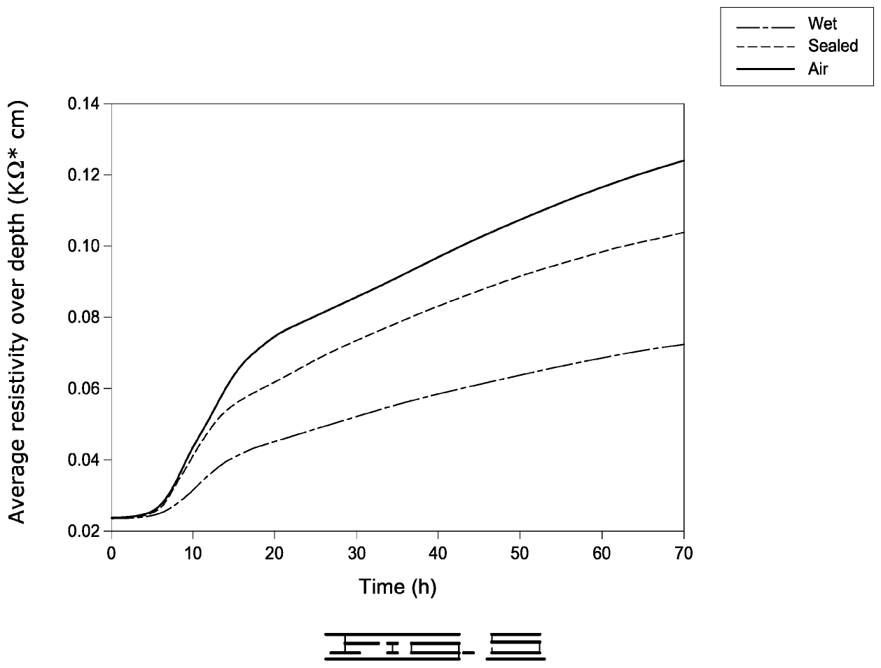

The average resistivity along the sample depth of the three curing methods is shown in FIG. 5. Every curing method showed growth in resistivity over time and the resistivity curve of different curing methods presented a similar shape. The similar shape of resistivity growth was likely caused by hydration reactions and consumption of water over time. As the hydration reaction proceeded, free water was consumed to form hydration products as the mixture changed from a slurry to a solid. As a result, the average resistivity of the mortar specimen increased with hydration. The air curing showed the highest resistivity followed by the sealed sample, and then the wet cured sample had the lowest resistivity. The difference in resistivity depended on several factors including the amount of moisture in the pores, ion concentration in the pores and the tortuosity of the pore network.

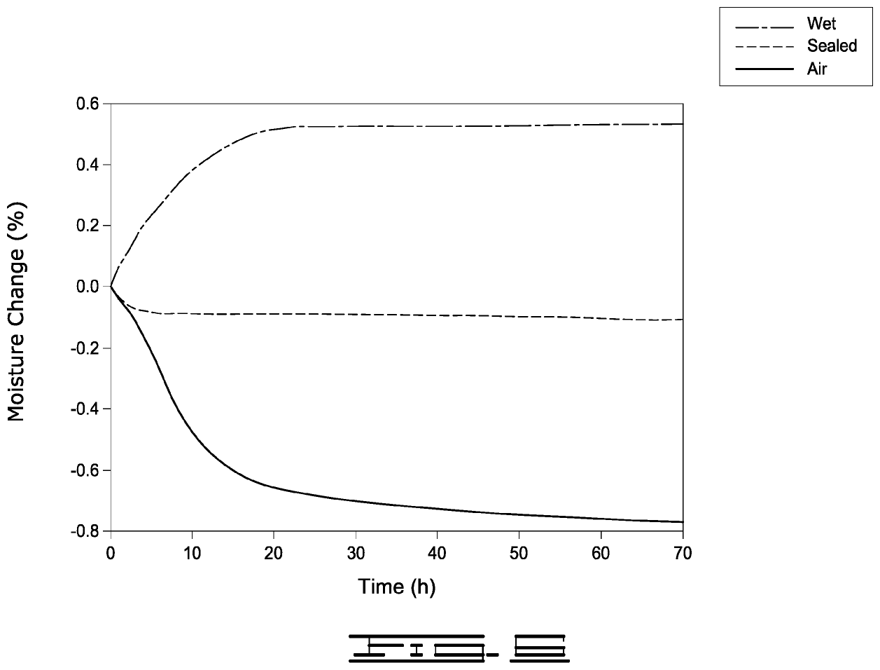

FIG. 6 shows the percentage of moisture change for the three curing methods. It shows that the wet curing sample had absorbed moisture within the first 20 h and the air curing sample had the most mass loss in the first 20 h. A slight moisture loss happened to the sealed sample during the first 8 h and then the mass was constant. In all samples, the mass change stopped after roughly 20 h. This was likely caused by the hydration process reducing the porosity and pore connectivity in the mortar. As stated above, the difference in resistivity between curing methods was at least partially caused by moisture differences between the samples. The moisture change curve supports this statement by showing that the air curing sample had the most moisture loss over time and the wet curing sample had moisture absorption over time.

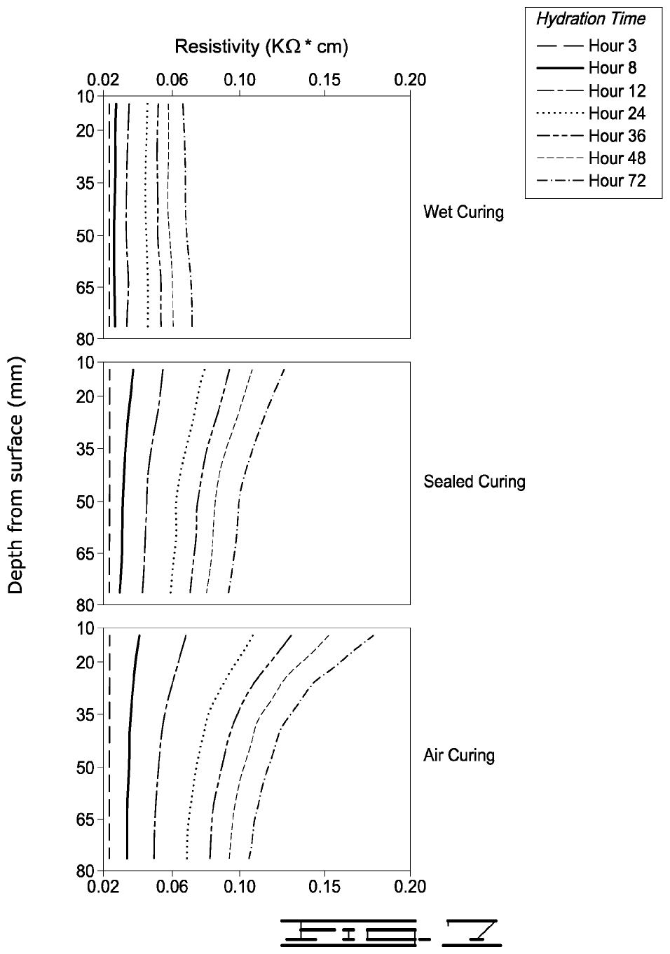

FIG. 7 shows the resistivity profile over the sample depth at different times. The wet curing sample had uniform resistivity over the depth. There is a resistivity gradient observed for the air cured and sealed cured samples. This gradient is first observed at about 8 h in the sealed and air cured sample and it continues to magnify over time. These same samples showed measurable moisture loss at 8 h in the drying experiments in FIG. 4. This loss of water likely contributed to the observed changes in resistivity. The higher resistivity at 12.7 mm compared to 76.2 mm is expected if there is more moisture loss at the surface of the sample.

The DOS of the wet curing specimen was expected to be higher than the other curing methods because of the extra moisture provided over time. This extra moisture promoted the hydration reaction and this reduced the porosity of the wet curing samples compared to the other methods. For the sealed sample, the moisture was consumed during the hydration and there was some evaporation over the first several hours of hydration. In the air curing sample, water was consumed during hydration and also lost to evaporation. Because of the loss of water, the air curing sample was expected to have the highest porosity and lowest DOS.

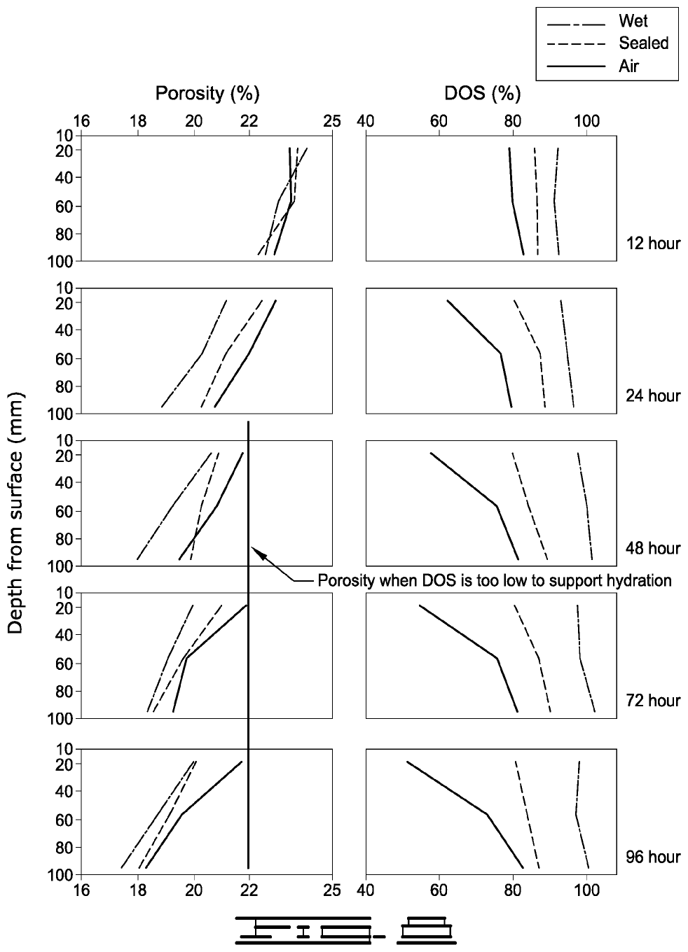

FIG. 8 shows how the porosity and DOS change over time. Each line on the figure represents a different curing method. At 12 h, the three curing methods had almost the same porosity over the depth, which indicates that the degree of hydration of the three curing methods is similar despite the difference in moisture content. It should be noted that after hydrating for 24 h, there was a porosity gradient in every sample with the furthest depth having the lowest porosity and the surface having the highest porosity. This higher porosity at the surface may have been caused by differences in bleeding. When comparing the results at each depth, the air cured sample had the highest porosity followed by the sealed cured sample, and the wet cured sample had the lowest porosity. This difference in porosity between the curing types was caused by the differences in the amount of moisture available for hydration. It should be noted that the air cured sample stopped showing a porosity change at 19 mm from the surface after 48 h. This might have been caused by the humidity not being high enough within the sample to sustain hydration.

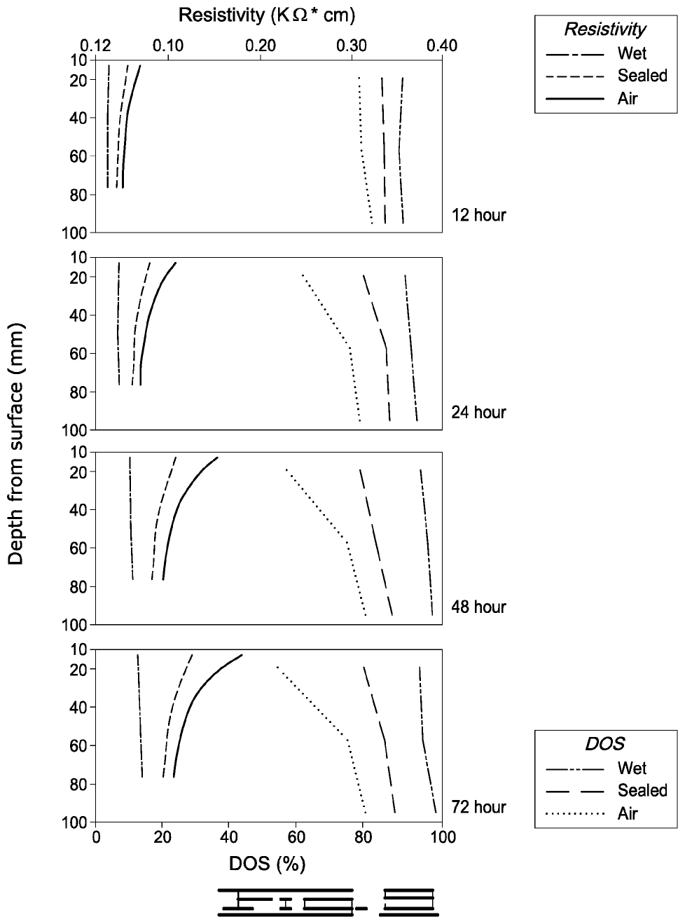

FIG. 9 shows the resistivity and DOS at 12, 24, 48, and 72 h of hydration over the sample depth for the three curing methods. The graph shows that the DOS and resistivity curves are correlated. For example, as the DOS begins to decrease, the resistivity increases. This means the resistivity value gives important insights into the moisture content inside the concrete. This also means that a resistivity value taken near the surface and one taken in the depths of the sample may be used as an indicator of the DOS and hence the moisture in the concrete. This may be used as a tool to measure the quality of the curing method applied to the concrete.

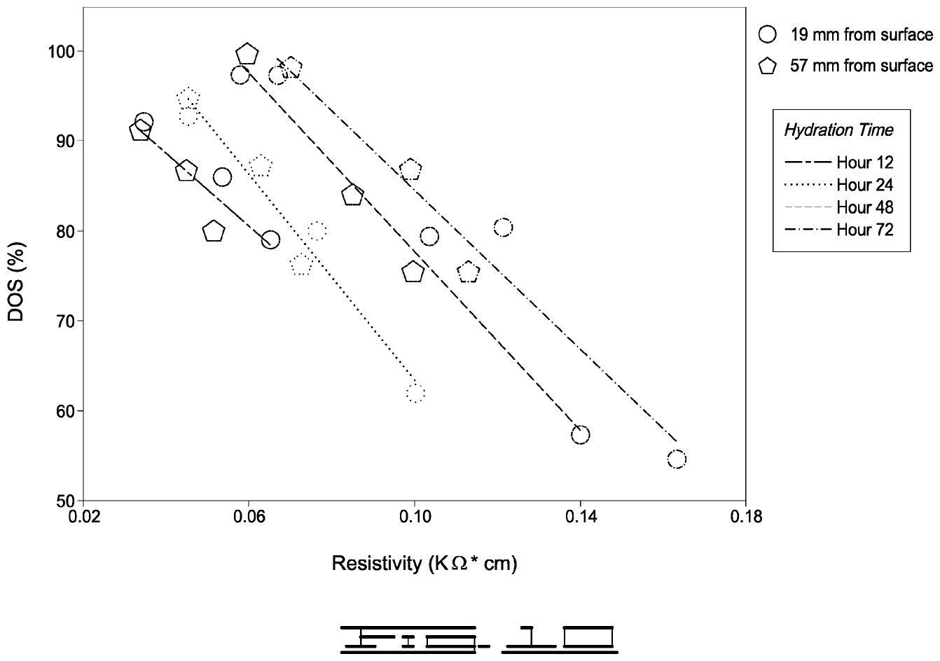

FIG. 10 shows that the resistivity and DOS can be assumed to be linearly related between 12 h and 72 h of hydration. The fitted curve between resistivity and DOS through linear regression at different times is shown in FIG. 10. The resistivity at each point was determined by linear interpolation of the two adjacent points that varied by +/−5 mm. FIG. 10 shows that the resistivity and DOS were linearly related between 12 h and 72 h of hydration. The fitted curve between resistivity and DOS through linear regression at different times is shown in FIG. 10 and this linear regression function can be used to predict the DOS value with resistivity measurements. The R square value is close to 1 for 24 h, 48 h, and 72 h, which illustrates the high accuracy of the linear relationship. These fitted curves can then be used to predict the DOS at any depth within the sample at a given time.

FIG. 11 shows the predicted DOS based on the resistivity measurements. The predicted DOS shows good agreement with the measured DOS value. This means using resistivity measurement can quantitatively compare the DOS created by different curing methods for a given set of materials. This may be used as a new tool to measure how water loss from hydration or drying correlate to the DOS or water content within a concrete sample. Since this moisture content during hydration is so important to the properties of the concrete, these resistivity measurements provide important insights into the material properties of the concrete.

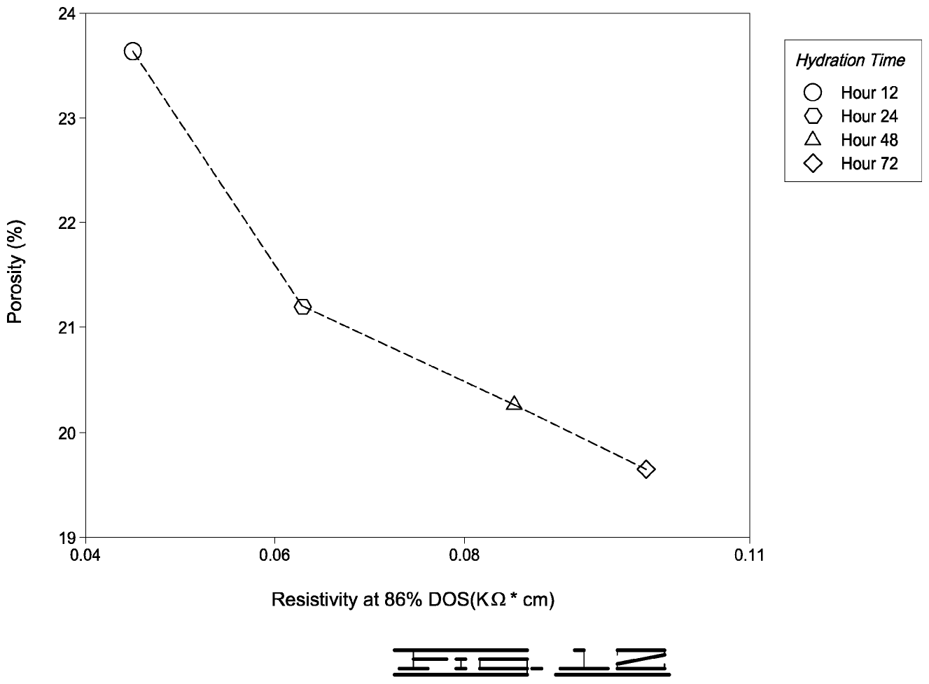

As seen in FIG. 10, as the hydration proceeds, the DOS versus the resistivity curve shifts to the right and the slope of the curve decreases, which indicates that resistivity becomes more and more sensitive to DOS change as hydration proceeds. This is likely because the formation of hydration products decreases the porosity and increases the resistance of electron mobility. FIG. 12 shows the measured porosity versus the resistivity at 86% DOS at different hydration times. This porosity and resistivity were chosen from the sealed curing specimen and a DOS of 86% was chosen because it would ensure hydration is continuing. Because a constant DOS is used, the plot shows how the porosity would change with resistivity at a fixed DOS. This means that as the hydration reaction proceeds, the porosity decreases, and the resistivity increases. The relationship seems to be bilinear between 12 h and 72 h. This change in the shape of the curve could be caused by differences in pore solution chemistry or the connectivity of the pore structure at different times. This is an important finding because it shows the ability of resistivity to give insights into the pore structure properties of mortar as it hydrates.

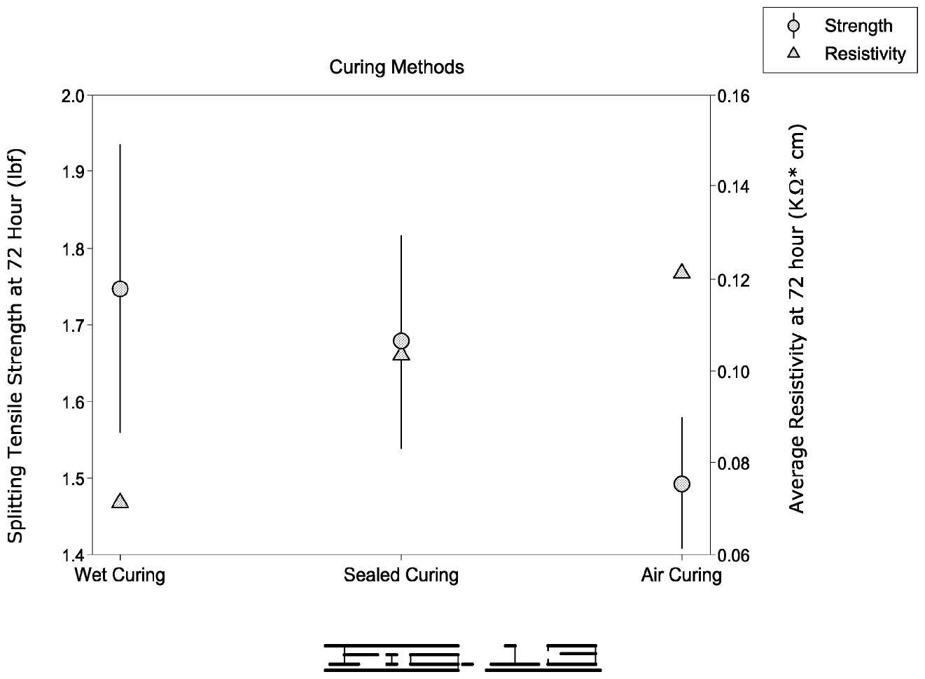

FIG. 13 shows the splitting tensile strength at 72 h for the three different curing methods in comparison to the average resistivity at the same time and the same depth. The results show that the resistivity was inversely proportional to the splitting tensile strength of the concrete. For example, the wet curing sample exhibited the highest splitting tensile strength and the lowest resistivity and the air cured sample showed the lowest splitting tensile strength and the highest resistivity. These differences in strength and resistivity are likely caused by differences in the DOS during hydration. This means that the resistivity can give insights into the tensile strength of the concrete.

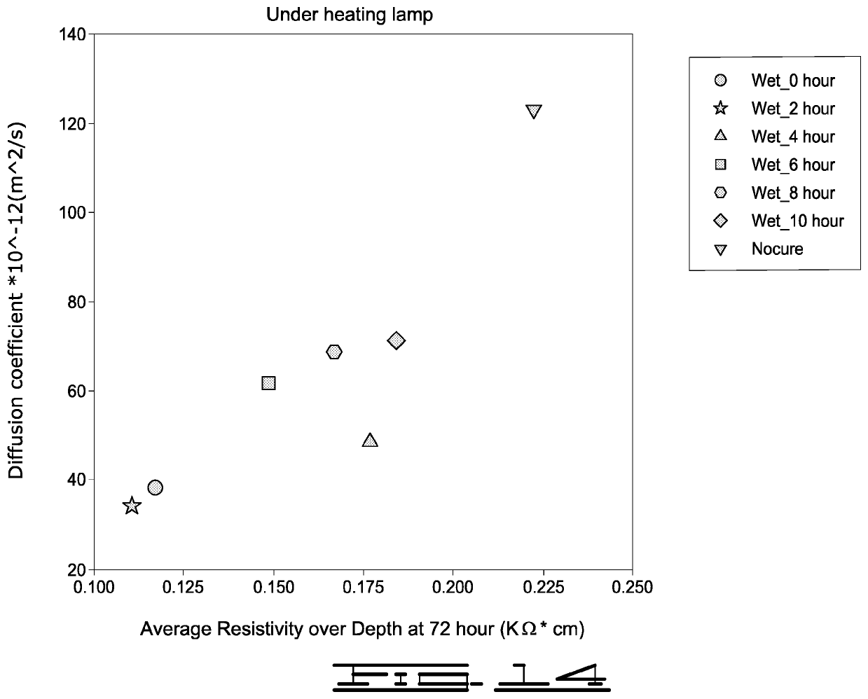

FIG. 14 portrays a graph displaying the diffusion coefficient as measured on mortar as it relates to the average resistivity over depth at 72 h from a comparable concrete mixture. The time next to the individual data points is the time when the wet curing was applied to the surface of the samples. The samples were cured under a heat lamp to increase the moisture loss of the samples. As can be seen from the graph, as the time before applying the wet curing increased, the average resistivity over depth at 72 h as well as the diffusion coefficient increased.

This work compares the resistivity of air, sealed, and wet curing at different depths in a sample. The results suggest that the resistivity can be correlated to the DOS, porosity, and tensile strength of the sample between 12 h and 72 h. This means that resistivity measurements during this period can be used to determine the uniformity and effectiveness of different curing methods and provide insights into the uniformity of the hydration over the depth of the sample. This information can provide insights into the porosity and ultimately the strength of the samples at different depths by making real time measurements that are minimally intrusive. These measurements can be used in either the field or the lab to inexpensively evaluate concrete quality in almost real time. For example, this technique can be used to determine how sensitive a concrete mixture is to different curing practices. This technique can also be used in the field to use the resistivity gradient in the concrete to give insights into the quality of the real time curing and hydration of the concrete.

These results demonstrate how impedance and resistivity values can be correlated with other properties of curing concrete samples for a particular set of external conditions, concrete compositions, and curing methods, in order to assist in the interpretation of impedance data for a given concrete application. The results further demonstrate how different curing conditions, such as wet curing, sealed curing, and air curing, can be used to adjust or correct the curing characteristics, based on the impedance correlations, to bring the curing concrete system within defined parameters for given chemical or physical properties. This makes the invention disclosed herein a valuable means for determining the quality of curing concrete samples, which helps in the field to guide curing practices. For example, based on the impedance gradient in a concrete sample, curing conditions may be modified to ensure that the impedance always stays below some critical value, as determined in respect to a given application.

The gradient information can also be used to compare the moisture loss of the material at the surface to the moisture loss of the material within the bulk of the sample. This is another useful way to apply this information in practice in that it does not require a correlation of the electrical impedance to a specific property.

It is to be understood that the foregoing examples do not limit the invention disclosed herein and may be altered or adjusted to suit the scale, composition, or other characteristics of a given concrete application according to methods known by those of ordinary skill in the field. For example, the number of sensors and the distance between the sensors may vary depending on the scale of the application. As the number of sensors and the distance between the sensors vary, the resistance or impedance may be calculated based on the distance between the sensors according to known methods.

The above disclosure, and embodiments thereunder, are exemplified by methods and systems defined by the following numbered paragraphs:
1. A method for monitoring the properties of a curing concrete mixture so as to produce a hardened concrete having at least one predetermined property, the method comprising:
   (a) receiving electrical impedance data representative of impedance across all or a selected portion of the curing concrete mixture, wherein the electrical impedance data is received during the curing of the curing concrete mixture and is representative of changes in impedance over a first period of time from an initial time to a second time;
   (b) analyzing the electrical impedance data for the existence of an electrical impedance gradient with respect to depth for the curing concrete mixture to produce an electrical impedance gradient for the first period of time; and
   (c) correlating the electrical impedance gradient for the first period of time to the amount of moisture loss at a plurality of locations within concrete mixture during the first period of time to produce a moisture correlation.
2. The method of paragraph 1, further comprising:
   receiving temperature data representative of temperature across all or a selected portion of the curing concrete mixture, and
   wherein correlation of the electrical impedance gradient includes correlating the data for temperature variations in the curing concrete mixture.
3. The method of paragraphs 1 or 2, further comprising:
   determining a change to the set of curing conditions based on the moisture correlation, wherein the change in the set of curing conditions results in an electrical impedance gradient for a second period of time from the second time to a third time, and wherein the electrical impedance gradient for the second period of time is closer to a predetermined gradient than the electrical impedance gradient for the first period of time; and
   applying the change to the set of curing conditions to the curing concrete mixture.
4. The method of paragraph 3, wherein the step of applying the change comprises adjusting the rate of moisture change in the curing concreter and adjusting the rate of heat change in the curing concrete.
5. The method of any of paragraphs 1 to 4, wherein the step of correlating the electrical impedance gradient for the first period of time comprises determining at least one first expected property of the curing concrete mixture or hardened concrete based on the electrical impedance gradient for the first period of time, and wherein the first expected property includes one or more of moisture content, porosity, permeability and strength.
6. The method of paragraph 5, wherein the step of correlating the electrical impedance gradient for the first period of time comprises comparing the at least one first expected property of the curing concrete mixture with at least one predetermined property of the curing concrete mixture or hardened concrete wherein the predetermined property includes one or more of a predetermined moisture content, a predetermined porosity, a predetermined permeability and a predetermined strength, and wherein the change in the set of curing conditions is configured to bring a second expected property based on the electrical impedance gradient for the second period of time closer to the predicted property than the first expected property.
7. The method of any of paragraph 6, wherein the predetermined property includes all of porosity, permeability, predetermined degree of saturation, predetermined diffusion coefficient, and the predetermined strength, and the first expected property includes all of porosity, permeability, degree of saturation, diffusion coefficient, and the strength.
8. The method of any of paragraphs 1 to 7, wherein the steps are repeated for the electrical impedance gradient for the second period of time.
9. The method of paragraph 1 or 2, further comprising:
   defining a predetermined electrical impedance gradient with respect to depth for the curing concrete mixture; and
   wherein the step of correlating the electrical impedance gradient comprises determining if the electrical impedance gradient for the first period of time meets the predetermined gradient.
10. The method of paragraph 9, further comprising:
   when the electrical impedance gradient for the first period of time does not meet the predetermined gradient, determining a change in the set of curing conditions which will result in a change to the gradient so as to meet the predetermined gradient; and
   applying the change in the set of curing conditions to the curing concreter mixture.
11. The method of any of paragraph 10, wherein the step of applying the change comprises at least one of adjusting the rate of moisture change in the curing concrete and adjusting the rate of heat change in the curing concrete.
12. A system for monitoring the properties of a curing concrete mixture so as to produce a hardened concrete having at least one predetermined property, the system comprising:
   a) the curing concrete mixture, which will cure into the hardened concrete under a set of curing conditions;
   b) one or more electrical impedance sensors, the one or more sensors comprising a plurality of electrically conductive components distributed at one or more respective locations within the concrete mixture so as to pass an electrical current across respective portions of the concrete mixture, wherein the sensor is configured to produce electrical impedance data based on the passing of the electrical current;
   c) a processing unit connected to the one or more electrical impedance sensors and configured to receive and process the electrical impedance data, wherein the processing unit processes the electrical impedance data by a data processing program configured to perform the following operational steps;
      receiving electrical impedance data representative of impedance across all or a selected portion of the curing concrete mixture, wherein the electrical impedance data is received during the curing of the curing concrete mixture and is representative of changes in impedance over a first period of time from an initial time to a second time;
      analyzing the electrical impedance data for the existence of an electrical impedance gradient with respect to depth for the curing concrete mixture to produce an electrical impedance gradient for the first period of time; and
      correlating the electrical impedance gradient for the first period of time to the amount of moisture loss at a plurality of locations within concrete mixture during the first period of time to produce a moisture correlation.
13. The system of paragraph 12, wherein the system further comprises one or more temperature sensors distributed at one or more respective locations within the concrete mixture, wherein the temperature sensors measure temperature at the one or more respective locations to produce temperature data, and the data processing program receives the temperature data, and wherein the correlation of the electrical impedance gradient includes correlating the data for temperature variations in the curing concrete mixture, such as by adjusting or correcting the electrical impedance gradient based on temperature difference of measured temperatures at two or more of the locations.
14. The system of paragraph 12 or 13, further comprising a material placed in contact with a surface of the curing concrete mixture to adjust a rate of change of moisture content.
15. The system of any of paragraphs 12 to 14, wherein the data processing program is further configured to perform the following operational step:
   determining a change to the set of curing conditions based on the moisture correlation, wherein the change in the set of curing conditions results in an electrical impedance gradient for a second period of time from the second time to a third time, and wherein the electrical impedance gradient for the second period of time is closer to a predetermined gradient than the electrical impedance gradient for the first period of time.
16. The system of paragraph 15, wherein the step of correlating the electrical impedance gradient for the first period of time comprises determining at least one first expected property of the curing concrete mixture or hardened concrete based on the electrical impedance gradient for the first period of time, and wherein the first expected property includes one or more of moisture content, permeability and strength.
17. The system of paragraph 16, wherein the step of correlating the electrical impedance gradient for the first period of time comprises comparing the at least one first expected property of the curing concrete mixture with at least one predetermined property of the curing concrete mixture or hardened concrete wherein the predetermined property includes one or more of a predetermined moisture content, a predetermined porosity, a predetermined permeability and a predetermined strength, and wherein the change in the set of curing conditions is configured to bring a second expected property based on the electrical impedance gradient for the second period of time closer to the predicted property than the first expected property.
18. The system of paragraph 17, wherein the predetermined property includes all of porosity, permeability, predetermined degree of saturation, predetermined diffusion coefficient, and the predetermined strength, and the first expected property includes all of porosity, permeability, degree of saturation, diffusion coefficient, and the strength.
19. The system of any of paragraphs 12 to 14, further comprising:
   defining a predetermined electrical impedance gradient with respect to depth for the curing concrete mixture; and
   wherein the step of correlating the electrical impedance gradient comprises determining if the electrical impedance gradient for the first period of time meets the predetermined gradient.
20. A method for monitoring the properties of a curing concrete mixture so as to produce a hardened concrete having at least one predetermined property, the method comprising:
   defining at least one predetermined property of the curing concrete mixture or hardened concrete, wherein the predetermined property includes one or more of a predetermined moisture content, a predetermined porosity, a predetermined permeability, and a predetermined strength;
   receiving electrical impedance data representative of impedance across all or a selected portion of the curing concrete mixture, wherein the electrical impedance data is received during the curing of the concrete mixture and is representative of changes in impedance over time;
   correlating the electrical impedance data with a preexisting set of electrical impedance correlation data so as to determine at least one expected property of the curing concrete mixture or hardened concrete based on changes to the electrical impedance data over time, wherein the expected properties include one or more of the moisture content, the permeability, and the strength;
   determining if the expected property meets the predetermined property;
   when the expected property does not meet the predetermined property, determining a change in the set of curing conditions which will result in a change to the expected property so as to meet the predetermined property; and
   applying the change in the set of curing conditions to the curing concrete mixture.

Those skilled in the field will understand that the invention disclosed herein can be adapted for use in curing concrete systems of varying sizes, shapes, compositions, environmental conditions such as temperature, pressure, or humidity, and with varying physical orientations and placements of the rods or sensors. It will occur to those skilled in the art which sizes, shapes, and compositions, which environmental conditions such as temperature, pressure, or humidity, and which number and physical orientation and placement of rods or sensors best suits a given curing concrete application.

What is claimed is:

1. A method for monitoring the properties of a curing concrete mixture so as to produce a hardened concrete having at least one predetermined property, the method comprising:
(a) receiving electrical impedance data representative of impedance across all or a selected portion of the curing concrete mixture, wherein the electrical impedance data is received during the curing of the curing concrete mixture and is representative of changes in impedance over a first period of time from an initial time to a second time;
(b) analyzing the electrical impedance data for the existence of an electrical impedance gradient with respect to depth for the curing concrete mixture to produce an electrical impedance gradient for the first period of time; and
(c) correlating the electrical impedance gradient for the first period of time to the amount of moisture loss at a plurality of locations within concrete mixture during the first period of time to produce a moisture correlation.

2. The method of claim 1, further comprising:
receiving temperature data representative of temperature across all or a selected portion of the curing concrete mixture, and
wherein correlation of the electrical impedance gradient includes correlating the data for temperature variations in the curing concrete mixture.

3. The method of claim 2, further comprising:
determining a change to the set of curing conditions based on the moisture correlation, wherein the change in the set of curing conditions results in an electrical impedance gradient for a second period of time from the second time to a third time, and wherein the electrical impedance gradient for the second period of time is closer to a predetermined gradient than the electrical impedance gradient for the first period of time; and
applying the change to the set of curing conditions to the curing concrete mixture.

4. The method of claim 3, wherein the step of applying the change comprises adjusting the rate of moisture change in the curing concrete and adjusting the rate of heat change in the curing concrete.

5. The method of claim 3, wherein the step of correlating the electrical impedance gradient for the first period of time comprises determining at least one first expected property of the curing concrete mixture or hardened concrete based on the electrical impedance gradient for the first period of time, and wherein the first expected property includes one or more of moisture content, porosity, permeability and strength.

6. The method of claim 5, wherein the step of correlating the electrical impedance gradient for the first period of time comprises comparing the at least one first expected property of the curing concrete mixture with at least one predetermined property of the curing concrete mixture or hardened concrete wherein the predetermined property includes one or more of a predetermined moisture content, a predetermined porosity, a predetermined permeability and a predetermined strength, and wherein the change in the set of curing conditions is configured to bring a second expected property based on the electrical impedance gradient for the second period of time closer to the predicted property than the first expected property.

7. The method of claim 6, wherein the predetermined property includes all of porosity, permeability, predetermined degree of saturation, predetermined diffusion coefficient, and the predetermined strength, and the first expected property includes all of porosity, permeability, degree of saturation, diffusion coefficient, and the strength.

8. The method of claim 7, wherein the steps are repeated for the electrical impedance gradient for the second period of time.

9. The method of claim 2, further comprising:
defining a predetermined electrical impedance gradient with respect to depth for the curing concrete mixture; and
wherein the step of correlating the electrical impedance gradient comprises determining if the electrical impedance gradient for the first period of time meets the predetermined gradient.

10. The method of claim 9, further comprising:
when the electrical impedance gradient for the first period of time does not meet the predetermined gradient, determining a change in the set of curing conditions which will result in a change to the gradient so as to meet the predetermined gradient; and
applying the change in the set of curing conditions to the curing concrete mixture.

11. The method of claim 10, wherein the step of applying the change comprises at least one of adjusting the rate of moisture change in the curing concrete and adjusting the rate of heat change in the curing concrete.

12. A system for monitoring the properties of a curing concrete mixture so as to produce a hardened concrete having at least one predetermined property, the system comprising:
a) the curing concrete mixture, which will cure into the hardened concrete under a set of curing conditions;
b) one or more electrical impedance sensors, the one or more sensors comprising a plurality of electrically conductive components distributed at one or more respective locations within the concrete mixture so as to pass an electrical current across respective portions of the concrete mixture, wherein the sensor is configured to produce electrical impedance data based on the passing of the electrical current;
c) a processing unit connected to one or more electrical impedance sensors and configured to receive and process the electrical impedance data, wherein the processing unit processes the electrical impedance data by a data processing program configured to perform the following operational steps;
receiving the electrical impedance data representative of impedance across all or a selected portion of the curing concrete mixture, wherein the electrical impedance data is received during the curing of the curing concrete mixture and is representative of changes in impedance over a first period of time from an initial time to a second time;
analyzing the electrical impedance data for the existence of an electrical impedance gradient with respect to depth for the curing concrete mixture to produce an electrical impedance gradient for the first period of time; and
correlating the electrical impedance gradient for the first period of time to the amount of moisture loss at a plurality of locations within concrete mixture during the first period of time to produce a moisture correlation.

13. The system of claim 12, wherein the system further comprises one or more temperature sensors distributed at one or more respective locations within the concrete mixture, wherein the temperature sensors measure temperature at the one or more respective locations to produce temperature data, and the data processing program receives the temperature data, and wherein the correlation of the electrical impedance gradient includes correlating the data for temperature variations in the curing concrete mixture.

14. The system of claim 13, further comprising a material placed in contact with a surface of the curing concrete mixture to adjust a rate of change of moisture content.

15. The system of claim 13, wherein the data processing program is further configured to perform the following operational step:
determining a change to the set of curing conditions based on the moisture correlation, wherein the change in the set of curing conditions results in an electrical impedance gradient for a second period of time from the second time to a third time, and wherein the electrical impedance gradient for the second period of time is closer to a predetermined gradient than the electrical impedance gradient for the first period of time.

16. The system of claim 15, wherein the step of correlating the electrical impedance gradient for the first period of time comprises determining at least one first expected property of the curing concrete mixture or hardened concrete based on the electrical impedance gradient for the first period of time, and wherein the first expected property includes one or more of moisture content, porosity, permeability and strength.

17. The system of claim 16, wherein the step of correlating the electrical impedance gradient for the first period of time comprises comparing the at least one first expected property of the curing concrete mixture with at least one predetermined property of the curing concrete mixture or hardened concrete wherein the predetermined property includes one or more of a predetermined moisture content, a predetermined porosity, a predetermined permeability and a predetermined strength, and wherein the change in the set of curing conditions is configured to bring a second expected property based on the electrical impedance gradient for the second period of time closer to the predicted property than the first expected property.

18. The system of claim 17, wherein the predetermined property includes all of porosity, permeability, predetermined degree of saturation, predetermined diffusion coefficient, and the predetermined strength, and the first expected property includes all of porosity, permeability, degree of saturation, diffusion coefficient, and the strength.

19. The system of claim 13, further comprising:
defining a predetermined electrical impedance gradient with respect to depth for the curing concrete mixture; and
wherein the step of correlating the electrical impedance gradient comprises determining if the electrical impedance gradient for the first period of time meets the predetermined gradient.

20. A method for monitoring the properties of a curing concrete mixture so as to produce a hardened concrete having at least one predetermined property, the method comprising:
defining at least one predetermined property of the curing concrete mixture or hardened concrete, wherein the predetermined property includes one or more of a predetermined moisture content, a predetermined porosity, a predetermined permeability, and a predetermined strength;
receiving electrical impedance data representative of impedance across all or a selected portion of the curing concrete mixture, wherein the electrical impedance data is received during the curing of the concrete mixture and is representative of changes in impedance over time;
correlating the electrical impedance data with a preexisting set of electrical impedance correlation data so as to determine at least one expected property of the curing concrete mixture or hardened concrete based on changes to the electrical impedance data over time, wherein the expected properties include one or more of the moisture content, the porosity, the permeability, and the strength;
determining if the expected property meets the predetermined property;
when the expected property does not meet the predetermined property, determining a change in the set of curing conditions which will result in a change to the expected property so as to meet the predetermined property; and
applying the change in the set of curing conditions to the curing concrete mixture.
